(19) United States
(12) Patent Application Publication
(10) Pub. No.: US 2012/0269852 A1
(43) Pub. Date: Oct. 25, 2012

(54) Adjuvated Influenza Vaccine and Use Thereof

(76) Inventors: Alexander J. Kersten, Weesp (NL); Sara Ann Jackson, Pittsburgh, PA (US)

(21) Appl. No.: 13/382,639

(22) PCT Filed: Jul. 6, 2010
(86) PCT No.: PCT/EP10/59666

§ 371 Date: Jun. 1, 2012

Related U.S. Application Data
(60) Provisional application No. 61/223,889, filed on Jul. 8, 2009.
(60) Provisional application No. 61/226,345, filed on Jul. 17, 2009.

(30) Foreign Application Priority Data
Jul. 8, 2009 (EP) 09164938.4
Jul. 17, 2009 (EP) 09165769.2

(51) Int. Cl. A61K 39/145; A61P 37/04; A61P 31/16

(52) U.S. Cl. 424/210.1; 424/209.1

(57) ABSTRACT

A viral vaccine, specifically an influenza vaccine, comprises a combination of a component (a1) represented by a detoxified or non-toxic mutant of subunit A of an AB type exotoxin, and a component (a2) represented by at least one substance selected from the group consisting of metal salts and mineral salts, in association with a viral immunogen.

### FIELD OF THE INVENTION

The present invention relates to a viral vaccine comprising adjuvant and viral immunogen. The viral vaccine is particularly suitable for more specifically providing an influenza vaccine comprising adjuvant and influenza specific immunogen. The present invention provides for a new adjuvant concept used in the context of viral vaccines. It is useful for treating animals and in particular humans, and is suitable for seasonal and especially pandemic vaccines.

### DESCRIPTION OF THE BACKGROUND ART

Virus infection has been established and remains as a serious animal and human affliction. Especially in case of influenza, the virus can cause localized epidemics and global pandemics of acute infections. There is an ongoing desire to develop a virus vaccine prepared for a potential epidemic or even pandemic. Not only in emergency situations, but also under normal situations such as seasonal influenza prevention and in particular for human patients with weakened immune system like elderly people, immunization may be insufficient using a viral vaccine containing only a viral-specific immunogen as an active agent in an attempt to provide immunogenicity. Insufficient immogenicity may not be the only limitation of currently available vaccines. The necessity of a high antigen content per vaccine dose may also pose a problem. Especially during a pandemic, the need for a relatively high antigen content would limit the number of doses available for a given amount of producible viral antigen.

Attempts have been made, and commercially available virus vaccines have been developed, by the inclusion of adjuvants into vaccines with a view to stimulate the immune system and to augment immune response. Among the high variability of existing adjuvant classes and, still within each of the different adjuvant classes as well as adjuvant substances per class, for example inorganic materials such as metal salts and mineral salts, organic substances such as oil-based materials, lipids, emulsions, liposomes, proteosomes, lipopolysaccarides and other polysaccarides, toll-like receptors, toxins and many others have been contemplated and partly assessed in experimental investigations. Within the aforementioned general classes and still across individual members of each class, there exist a widespread and diverse heterogeneity of different substances and compounds. Owing to this heterogeneity among such different classes, adjuvanticity of substances and compounds is not generally predictable. Further, adjuvant function may be compromised by adverse side effects and risks. The use of an adjuvant—even for well known adjuvants that has extensively been used in vaccines for use in man as in the case of aluminum hydroxide—as an antigen-sparing mechanism, especially in threats of epidemics and pandemics, thus continuous to be scrutinized due to unknown potential systemic and local adverse side effects.

The complex nature of providing adjuvanting effect is reflected for example in the classes of bacterial toxin based adjuvants proposed for vaccines with virus antigens, e.g. by patent documents like WO2005/112991 A (proposing Shiga toxin B-subunit as adjuvant), WO2006/123155A (proposing the B-subunit of E.coli heat labile toxin as adjuvant) and WO2005/079841A (proposing B-subunit binding deficient exotoxins as adjuvants), as well as numerous further literature respectively reporting about various detoxified versions of bacterial toxins. In both WO2005/112991A and WO2006/123155A, alum adjuvanted, recombinant protein subunit vaccines are said to be notably poor inducers of immune responses.

The object of the present invention is to provide a viral vaccine with the capacity to elicit a satisfactory functional immune response in animals and particularly in humans, in balance with an acceptable or low level of side effects and reactogenicity.

### SUMMARY OF THE INVENTION

The present invention provides, in a first aspect, a viral vaccine comprising a combination of:

(a1)) detoxified or non-toxic mutant of subunit A of an AB type exotoxin, and

(a2) at least one substance selected from the group consisting of metal salts and mineral salts; and

(b) a viral immunogen.

In a second aspect, the present invention relates to an influenza vaccine comprising a combination of (a1) a detoxified or non-toxic mutant of subunit A of heat-labile enterotoxin (HLT), and (a2) at least one substance selected from the group consisting of metal salts and mineral salts; and an influenza-specific antigen.

The present invention also provides a use of a viral immunogen adsorbed on a metal salt or a mineral salt in combination with a detoxified or non-toxic mutant of subunit A of an AB type exotoxin, for the manufacture of a vaccine against the virus from which the viral immunogen is derived.

In a further aspect, the present invention relates to a particular adsorbate which comprises a viral immunogen and a detoxified or non-toxic mutant of subunit A of E. coli heat-labile enterotoxin (HLT), respectively adsorbed on aluminum hydroxide.

In particular, the present invention provides the following aspects, subject-matters and preferred embodiments, which respectively taken alone or in combination, contribute to solving the object of the present invention.

(1) Viral vaccine comprising a combination of:

(a1) detoxified or non-toxic mutant of subunit A of an AB type exotoxin, and

(a2) at least one substance selected from the group consisting of metal salts and mineral salts; and

(b) a viral immunogen.

The viral immunogen is specific to the virus for which the viral vaccine is designed and provided. It is preferred that the type and/or amount of each of the components (a1) and (a2) is effective to display adjuvant property. Component (a1) represents a toxicity-attenuated or toxicity-deficient, hence detoxified mutant form A of an AB type exotoxin, preferably of an AB5 type exotoxin. In a preferred embodiment component (a1) consists of detoxified or non-toxic mutant of subunit A only, i.e. being free of any form of subunit B. Although less preferred it is possible to associate subunit A with detoxified or non-toxic mutant of subunit B of the respective AB type exotoxin.

(2) The viral vaccine according to item (1), wherein said component (a1) is correspondingly derived from an AB type exotoxin selected from the group consisting of heat-labile enterotoxin (HLT), cholera toxin (CT), Shiga toxin (Stx, including Stx1 and Stx2), verotoxin, diphtheria toxin (DT), pertussis toxin (PT), butolinum toxin, Pseudomonas aeruginosa exotoxin A (ETA), and Ricin.

More preferably, component (a1) is detoxified or non-toxic mutant of subunit A of an AB type exotoxin selected from the group consisting heat-labile enterotoxin (HLT) and cholera toxin (CT), more preferably of HLT.

(3) The viral vaccine according to anyone of the preceding items, wherein said detoxified or non-toxic mutant of subunit A is derived from E. coli heat labile enterotoxin (HLT).

(4) The viral vaccine according to anyone of the preceding items, wherein said component (a1) is prepared by recombinant DNA methods.

(5) The viral vaccine according to anyone of the preceding items, wherein the subunit A of component (a1) has reduced ADP-ribosylating activity.

A particularly effective reduction in ADP-ribosylating activity is accomplished by a non-toxic variant by at least one mutation effective to reduce or diminish ADP-ribosylating activity.

(6) The viral vaccine according to item (5), wherein said detoxified or non-toxic mutant of subunit A to thereby reduce or diminish ADP-ribosylating activity is defined by protease-cleavage resistant, in particular trypsin-cleavage resistant subunit A.

(7) The viral vaccine according to anyone of the preceding items, wherein said detoxified or non-toxic mutant of subunit A contains substitution at location LTA-R192.

(8) The viral vaccine according to item (7), wherein said detoxified or non-toxic mutant of subunit A contains the substitution LTA-R192G.

(9) The viral vaccine according to anyone of the preceding items, wherein said component (a2) is selected from the group consisting of aluminum hydroxide, alum and aluminum phosphate, preferably is aluminum hydroxide.

(10) The viral vaccine according to item (9), wherein said component (a2) is aluminum hydroxide.

(11) The viral vaccine according to anyone of the preceding items, wherein said component (a2) is present in an amount limited to up to about 0.5 mg per vaccine dose. As a minimum, an amount sufficient to exert an adjuvant effect shall be used, effective to increase HAI assay titer relative to use of immunogen alone.

(12) The viral vaccine according to anyone of the preceding items, wherein said virus-specific immunogen of component (b) is not covalently bound to component (a1).

(13) Vaccine according to anyone of the preceding items, characterized by a vaccine type selected from the group consisting of subunit vaccine comprising purified surface antigen, split vaccine, inactivated whole virus vaccine, attenuated virus vaccine, recombinant protein vaccines, virosome vaccine, or virus-like-particle vaccine.

(14) The viral vaccine according to anyone of the preceding items, which contains, as immunogen component (b), any one selected from: inactivated or destructed whole virus particles, purified viral antigens, and virosomes containing antigens.

(15) The viral vaccine according to any one of the preceding items, which is an influenza vaccine comprising, as component (b), an influenza specific immunogen.

(16) The viral vaccine according to item (15), wherein the influenza immunogen component contains HA antigen which is limited by an amount of up to about 15 μg per strain per vaccine dose as measured by HA content, preferably below 15 μg, more preferably at most about 10 μg, particularly at most about 5 μg, suitably at about 2 μg HA.

(17) Influenza vaccine comprising a combination of

(a1) detoxified or non-toxic mutant of subunit A of heat-labile enterotoxin (HLT), and

(a2) at least one substance selected from the group consisting of metal salts and mineral salts;

and

(b) an influenza-specific antigen.

(18) The vaccine according to any one of items (15) to (17), wherein the component (a2) is aluminum hydroxide.

(19) The vaccine according to any one of items (15) to (18), which is a seasonal, epidemic or pandemic influenza vaccine.

(20) The vaccine according to any one of items (15) to (19), wherein the influenza specific immunogen is defined by the influenza antigen class H1, H2, H3, H5, H6, H7, N1, N2, N3 or N7, either alone or in combination, preferably influenza antigen class H1N1, H2N2, H3N2, H6N1, H7N3 or H7N7, preferably by class H3 or H5, particularly by H1N1 or H5N1.

(21) Use of a viral immunogen adsorbed on a metal salt or a mineral salt in combination with a detoxified or non-toxic mutant of modified subunit A of an AB type exotoxin, for the manufacture of a vaccine preparation or a vaccine kit against the virus from which the viral immunogen is derived.

The use results in a manufacture of the corresponding virus vaccine. The virus vaccine is suitable for use in medical prophylaxis. The use may include a fixed combination in a common dosage form (a common vaccine preparation), or an associated but separate administration of the immunogen adsorbed on a metal salt or a mineral salt on the one hand, and of the detoxified or non-toxic mutant of subunit A of an AB type exotoxin on the other hand. The latter case includes the provision of a kit of parts, respectively containing the separate ingredients.

(22) Use according to item (21), wherein said detoxified or non-toxic mutant of subunit A of AB type exotoxin, is co-adsorbed with the viral immunogen on the metal salt or a mineral salt.

(23) Use according to item (21) or (22), wherein the metal salt is aluminum hydroxide.

(24) Use according to anyone of items (21) to (23), wherein the immunogen is specific for influenza, for the manufacture of an influenza vaccine.

(25) Use of a viral vaccine according to anyone of items (1) to (20), or manufactured according to the use of anyone of items (21) to (24) for treating animals and humans, in particular humans.

(26) Use according to item (25), wherein the treatment includes intramuscular administration.

(27) A combination of a detoxified or non-toxic mutant of subunit A of an AB type exotoxin with an aluminium salt, for use in an immunization to provide an at least 3-fold, preferably an at least 5-fold antigen-sparing effect of at least one influenza-specific antigen against influenza virus.

The combination can be used as a fixed combination where both components are in one dosage form, or as a separate combination where both components are respectively used simultaneously, or sequentially but in associated order, but by way of separate dosage forms or by way of different parts of a kit.

As used herein the term “antigen-sparing effect” is a measure of the capacity of the combination to reduce the amount of respective antigen relative to the use of the corresponding type and amount of influenza-specific antigen which elicits about the same or a lower magnitude of immune response in the tested individuum (animal, specifically mammal, more specifically ferret and in particular human) against said at least one influenza-specific antigen of influenza virus but uses neither said subunit A nor said aluminium salt.

The immune response can be determined as GMT HI titer increase in a HAI assay (GMT=geometric mean titer, wherein the mean is calculated for the tested collective of e.g. 10 individuals).

(28) A combination of a detoxified or non-toxic mutant of subunit A of an AB type exotoxin with an aluminium salt, for use in an immunization to provide an at least 20-fold, preferably an at least 50-fold and more preferably an at least 100-fold higher immune response against at least one influenza-specific antigen of influenza virus, relative to the use of the corresponding type and amount of influenza-specific antigen which neither uses said subunit A nor said aluminium salt.

Likewise in the present use embodiment, the combination can be used as a fixed combination where both components are in one dosage form, or as a separate combination where both components are respectively used simultaneously, or sequentially but in associated order, but by way of separate dosage forms or by way of different parts of a kit.

And again, the immune response can be determined as GMT HI titer increase in a HAI assay (GMT=geometric mean titer, wherein the mean is calculated for the tested collective of e.g. 10 individuals).

(29) Adsorbate comprising

a viral immunogen and

a detoxified or non-toxic mutant of subunit A of E. coli heat-labile enterotoxin (HLT), respectively adsorbed to aluminum hydroxide.

(30) The adsorbate according to item (29), wherein said viral immunogen is defined by the presence of influenza specific antigens including influenza hemagglutinin (HA), preferably in addition influenza neuraminidase (NA).

(31) A method for immunizing a patient against virus infection, comprising the steps of:

administering to a person in need thereof a dose of immunogen of a viral against which the patient shall be immunized,

administering to said person a metal salt or a mineral salt, and

administering to said person a detoxified or non-toxic mutant of subunit A of an AB type exotoxin,

wherein the respective administration steps, independently from each other, are carried out simultaneously, within one or separate dosage forms, or sequentially in a prescribed order. For preferred embodiments of each of the administered components, reference is made to the items stated above.

### BRIEF DESCRIPTION OF THE DRAWINGS

FIG. 1 and FIG. 2 respectively show geometric mean and individual HI titers obtained after vaccination of ferrets by vaccines comprising virus antigen at various doses of HA antigen in non-adjuvanted or adjuvanted samples, including a comparison between samples using either Al(OH)₃ (Alum) only or non-toxic mutant LT-A only, or using a combination of Al(OH)₃ (Alum) and non-toxic mutant LT-A at variable adjuvant contents (mcg=μg). The results show a synergistic effect of a combination of the adjuvants of non-toxic mutant LT-A and Al(OH)₃ (Alum), and a significant and robust immune response even at relatively low adjuvant contents respectively.

### DETAILED DESCRIPTION OF THE INVENTION

The present invention is now described in more detail by preferred embodiments and examples, which are however presented for illustrated purposes only and shall not be understood as limiting the scope of the present invention in any way.

A combination of detoxified or non-toxic mutant of subunit A of an AB type exotoxin with a substance selected from metal salts and mineral salts surprisingly and unexpectedly provides a synergistic immune potentiating effect with respect to a viral immunogen. Remarkable adjuvanticity and immunogenicity properties have been demonstrated in a representative influenza infection model, notably in ferrets which closely mimic infection in humans and represents an established animal model for influenza virus infection (van der Laan et al, Expert Rev. Vaccines 7(6), 783-793 (2008)). The particular combination of components (a1) and (a2) defined above in association with a viral immunogen thus provides useful viral vaccines. In certain embodiments, the present invention can provide potent vaccine candidates against virus strains to which the human population is immunologically naive, such as H1N1, H5N1 and others. The present invention provides a new and valuable adjuvant concept; the virus vaccine is especially suited for seasonal, epidemic and even pandemic virus outbreaks, which is presently highly needed, for example for influenza vaccination concepts. Thus, the present invention can address the need to cope with differences in immunogenic potency of different virus strains, thereby addressing a difference in ‘need’ to increase the potency via the use of the particular adjuvant system.

Moreover, owing to the synergistically augmented immunogenicity, the viral vaccine provided by the present invention has a capacity to establish sufficient immune protection even in cases where, if desired, antigen content can be reduced and thus the number of vaccine doses available by a given produced amount of viral immunogen can be increased, which provides further benefits for example in cases of emergency, like epidemics and pandemics. The possibility of reducing the amount of either component (a1) or component (a2), or both, while achieving the same immune potentiating effect as if only one of the component was used absent of the combination, can further contribute to reduce the risk of adverse side effects.

Further, the viral vaccine according to the present invention, due to its significantly enhanced immunogenicity, is beneficially useful for immunizing individuals whose immune system is weakened, including e.g. elderly people.

It is believed that the combination of the selected “bacterial danger system” of component (a1) with a suitably chosen “delivery” system, or “co-acting” adjuvant mechanism using metal salts or mineral salts by component (a2), in the specific context of immune response raised by a viral immunogen contributes to reduce reactogenicity and toxicity normally present in natural bacterial toxins, without however reducing adjuvanticity but rather, to the surprising contrary, with enhancing an immune potentiating effect against the viral immunogen. The immune potentiating effect appears to be particularly effective in, but not limited to the context of intramuscular (i.m.) vaccination.

Without being bound to any theory, it may be assumed that the specific context in the combination of the active viral vaccine components gives rise to immune reactions acting in synergy. The immune system of the treated individual may display various immune response mechanisms with interacting pathways, presumably involving differentiating but interplaying viral antigen based and bacterial, yet detoxified “danger toxin” based responses combined with “delivery”-based phenomena, thereby leading to a peculiar interplay of short-term acting and long-term acting immune-specific factors.

Hence, the present invention provides a beneficial new adjuvant concept for animal and especially human vaccines in the specific context of, and directed to, viral antigen systems. The viral vaccine is, for example, beneficially embodied by including an influenza viral immunogen to thereby provide influenza vaccines, covering possible seasonal, epidemic and pandemic situations. Based on the synergistic effect of the selected co-adjuvants, virus vaccine efficacy can be obtained at lower than typical dose levels and thus may allow, if desired or considered necessary, lowering the concentration or amount of one or more of the vaccine components, optionally of the viral immunogen, per vaccine dose. More advantageously, each of the active components, i.e. the viral immunogen component (b), the exotoxin-derived adjuvant component (a1) and the metal or mineral salt adjuvant component (a2) can be reduced from normal or typical levels alone or in combination, which significantly increases the safety profile of the viral vaccines.

In a preferred embodiment, the active components and optionally further pharmaceutically acceptable excipients or carrier substances, are included in a common viral vaccine formulation, and such formulation can be provided in the form of unit doses. Alternatively, the viral vaccine according to the present invention is provided in the form of a vaccine preparation comprising separate formulations, for example in the form of a vaccine kit, for simultaneous or sequential co-administration independent of the route of administration for use in eliciting immune response to the viral immunogen component included in the vaccine preparation. A suitable embodiment is to include the viral immunogen together with the metal salt or mineral salt adjuvant into one formulation, and to include the exotoxin-derived adjuvant component (a1) in another separate formulation, or to provide both the metal salt or mineral salt and the exotoxin-derived component (a1) adjuvants in one formulation while incorporating the viral immunogen in a separate formulation, to be eventually used by respective co-administration, optionally further containing respectively suitable and pharmaceutically acceptable excipients, carriers and media in the corresponding separate formulations. The fixed inclusion of all active vaccine components within unit dosages represents a preferred embodiment, as a synergistic effect can be achieved while reducing a risk of wrong dosing. In particular embodiments, both the exotoxin-derived component (a1) and the viral immunogen are co-adsorbed by the metal salt or mineral salt of component (a2), which is supposed to be particularly effective to exert the synergistic immune potentiating effect.

As used herein the “component (a1)” means detoxified or non-toxic mutant of subunit A derived from an AB type of exotoxin. In particular preferred embodiments, “component (a1)” means that a corresponding wild-type subunit A has one or more mutations which renders the subunit A protease resistant. Specifically, a preferred detoxified form is such that subunit A contains, compared to wild-type sequence, at least one mutation effective to reduce or diminish ADP-ribosylating activity, thereby becoming protease-resistant. Due to a lack or inhibition of a protease function, subunit A is not cleaved or cleaved to a substantially reduced extend of exerting toxic effects. Said subunit A is preferably detoxified by recombinant means to introduce appropriate mutations. Although it is in principle possible that B-subunit is associated with such A-subunit, for example using a toxicity-attenuated or -deficient subunit B of the respective an AB type of exotoxin, component (a1) is preferably free of any B-subunit, because subunit B associated reactions may increase the risk of reactogenicity and toxicity, without however gaining or even enhancing an appreciable adjuvant effect. It has been found that subunit B of an exotoxin does not provide a noticeable adjuvant effect and therefore should be better avoided in the subunit-A component (a1).

The term “adjuvant” used herein means a substance contained in the viral vaccine which can contribute to, and preferably improves the immune response of an individual such as animal and especially human after vaccine administration. An “adjuvant property” or “adjuvanticity” can be evaluated by measuring antibody levels as for example in an HAI assay and is indicated when the antibody levels are increased relative to use of viral immunogen alone at otherwise the same conditions.

The term “viral immunogen” used herein means a virus-derived substance causing immune reaction of the body of an individual to be treated. It may include a humoral immune response and cell-mediated immune response, respectively specific for the virus from which the viral immunogen derives. The viral immunogen typically comprises a viral antigen, against which the immune response shall be generated, suitably a viral surface antigen and more specifically a viral surface protein. The viral immunogen component is thus distinct e.g. from endotoxic antigens, bacterial endotoxin, and exotoxins. The viral antigen can be presented in various forms, and correspondingly the vaccine type can be selected, depending on the viral immunogen preparation, from the group consisting of subunit vaccine, split vaccine, inactivated whole virus vaccine, attenuated virus vaccine, recombinant protein vaccine, virosome vaccine, virsi-like-particle vaccine, and similar vaccine forms known to those skilled in the art, as well as further developed forms. The vaccine according to the present invention is preferably specific to the viral immunogen, notably the viral antigen, and thus is preferably absent from non-viral antigens other than component (a1), especially absent from bacterial antigens different from component (a1).

The term “about” used herein generally means an acceptable tolerance range of ±10% around the corresponding referenced value and/or measured value relevant for the respective context.

The component (a1) can be derived from correspondingly toxicity-attenuated or -deficient forms, hence detoxified or non-toxic mutant forms of subunit A of exotoxins selected from the group consisting of heat-labile enterotoxin (HLT), cholera toxin (CT), Shiga toxin (Stx, including Stx1 and Stx2), verotoxin, diphtheria toxin (DT), pertussis toxin (PT), butolinum toxin, or Pseudomonas aeruginosa exotoxin A (ETA) and Ricin. The combinatory provision and use of mutant forms of subunit A of heat-labile enterotoxin (HLT) of Escherichia coli to render it protease-resistant has lead to significant synergistic effects and thus are particularly preferred. Explanations as understood from the obtained results and as disclosed herein render it credible that corresponding combinatory effects can be achieved also when using subunit A of other exotoxins, notably those enumerated above, in combination with components (a2) and (b).

In certain embodiments with respect to component (a1), the toxicity-attenuated or toxicity-deficient, hence detoxified or non-toxic mutant form of an AB type exotoxin, to be combined with the co-adjuvant of metal or mineral salt, consists of detoxically modified subunit A only, preferably lacking subunit B. Subunit A of the AB type exotoxins disclosed herein, lacking B-subunit, can be formed on the basis of the respectively known wild-type exotoxin sequence, or partial sequences thereof, or modified sequences thereof having for example at least 90%, preferably at least 95% and more preferably at least 98% homology to the respective wild-type sequence, yet being detoxified or non-toxic mutant forms as disclosed herein. In preferred embodiments, detoxified or non-toxic mutant of subunit A is produced by recombinant DNA technology such as site-directed mutagenesis, especially by substitutions, deletions, insertions or additions of amino acids, wherein sequence modifications may show the aforementioned sequence homology to the wild-type sequence. The modified form itself preferably possess adjuvant property. Although subunit B may be present in association with the relevant detoxified or non-toxic mutant of subunit A, and in such optional case the subunit B in turn may be modified, the subunit A component (a1) is preferably free of subunit B, because it has been found to be ineffective in exerting an adjuvant effect by itself, but rather increases a risk of reactogenicity to a substantial extend.

According to a particularly preferred embodiment, the detoxified or non-toxic mutant of subunit A lacks ADP-ribosylating activity. Lack of ADP-ribosylating activity can be achieved for example by rendering the enzyme-sensitive, in particular the trypsin-sensitive, sequence location insensitive, to thereby effectively reduce reactogenicity and toxicity.

In certain embodiments, useful toxicity-attenuated or toxicity-deficient forms of an AB type exotoxin according to component (a1), to be combined with the metal salt or mineral salt, is selected from, without being limited to, the group consisting of:

detoxical modifications of A-subunits of CT and particularly of LT, selected e.g. from:

modification in the enzyme/trypsin-sensitive cleavage site of the A-subunit, 187-CGNSSRTITGDTC-199, to the effect that the subunit A lacks of ADP-ribosylating activity;

substitution R192, preferably R192G (Dickinson and Clements, Infect Immun. 63, 1617-23 (1995); Cheng et al., Vaccine 18, 38-49 (2000); WO 96/06627);

substitution H44A (Hagiwar et al., Vaccine 19, 2071-79 (2001);

substitution S63K or S63Y of the A-subunit (WO93/13202; Giannelli et al., Infect Immun. 65, 331-34 (1997); Pepoloni et al., Expert Rev. Vaccines 2, 285-93 (2003); Stevens et al., Infect Immun. 67, 259-65 (1999);

substitution A72R (Neidleman et al., Immunology 101, 154-60 (2000); Pepoloni et al., Expert Rev. Vaccines 2, 285-93 (2003));

substitutions in LT A-subunit defined by S61F, A69G, E112K, and H44R (Cheng et al., Vaccine 18, 38-49 (2000));

LT A-subunit substitutions V53D, R7K, V97K and Y104K (Stevens et al., Infect Immun. 67, 259-65 (1999));

LT A-subunit substitutions T50G or T50P and V53G or V53P (Neidleman et al., Immunology 101, 154-60 (2000); Verweij et al., Vaccine 16, 2069-76 (1998));

the detoxically modifying substitutions of CT and LT disclosed in W093/13202;

substitution P106S of CT subunit A (Pizza et al., Vaccine 19, 2534-41 (2001); WO93/13202);

substitutions in subunit A (S1) of the pertussis toxin disclosed in EP0322533A1 and EP 0322115A2; and

non-toxic A subunits of Ricin toxin RTA (Allen et al., Yeast 22(16), 1287-97 (2005).

Besides the above defined forms and amino acid substitutions, the subunit A may have wild-type sequences of the corresponding exotoxin, or may contain further modifications including, for example, additions, deletions, substitutions etc while showing for example at least 90%, preferably at least 95% and more preferably at least 98% homology to the respective wild-type sequence, or a partial sequence including the above defined modifications, with the proviso that the resulting exotoxin form maintains toxicity-attenuation or -deficiency as well as adjuvanticity in combination with the metal salt or mineral salt. Furthermore, in the above listed examples of sequence modifications, it is referred to the normal sequence alignment position of the corresponding wild-type sequence in order to identify the position of substitution. These indications shall however be understood in the sense that the final detoxified or non-toxic mutant of subunit A may contain further sequence modifications like sequence deletions and additions which will change the position with respect to the corresponding wild-type position.

With respect to wild-type sequences, it can be referred to known forms of the respective AB type exotoxins described in the literature.

In a particularly preferred embodiment, component (a1) is subunit A, characterized by substitution at R192 and in particular by the specific substitution R192G. Further preferred, this form is derived from heat-labile enterotoxin (HLT) of Escherichia coli.

As noted, the detoxified or non-toxic mutant of subunit A of component (a1) is derived in particularly preferred embodiments from Escherichia coli heat labile enterotoxin (HLT). As further specified above with respect to modified forms of enterotoxin generally, detoxic modifications being defined by an enzyme-cleavage resistant sequence, in particular trypsin-cleavage resistant sequence, free of subunit B, are beneficially effective, preferably the specific mutant form LT A-R192 and more preferably LT A-R192G. Besides such detoxifying modifications, further included are the sequence modifications and substitutions defined above and applied to LTA, including partial sequences and substitutions, deletions, additions showing for example at least 90%, preferably at least 95% and more preferably at least 98% homology to the respective wild-type sequence, or a partial sequence including the above defined modifications, with the proviso that the resulting form maintains toxicity-attenuation or -deficiency as well as adjuvanticity in combination with the metal salt or mineral salt.

In preferred embodiments, it is ensured that component (a1) is free, more preferably entirely free of wild type enterotoxin (HLT), which can be ensured by component (a1) being prepared by recombinant DNA methods.

In the above-referenced forms of component (a1), lack of ADP-ribosylating activity can be assayed by known methods, for example using the NAD-arginine ADP-ribosyl transferase assay (Moss et al., 1993, J. Biol. Chem. 268:6383-6387) or an equivalent assay, see also Giannelli et al. (1997), supra, and Stevens et al. (1999), supra. Further, ADP-ribosylating activity, or confirming lack thereof, can be commonly determined by measuring the cAMP-accumulation in Caco2-cells. Cytotoxicity, or confirming lack thereof, can be determined by morphological changes to CHO, HT29 or especially Y1 cells (Spangler, Microbiol. Review 56, 622-47 (1992)). For example, enterotoxicity, or confirming lack thereof, can be determined by feeding adult BALB/c mice 1-250 μg of component (a1), harvesting intestines after several hours and determine water accumulation by weight and calculating the gut to carcass ratio (Cheng et al., supra). Alternatively, enterotoxicity, or confirming lack thereof, can be determined by injection of mutant and wild-type toxins into isolated ileal loops of rabbits and subsequent determination of fluid accumulation (Giannelli et al., 1997, supra).

It is preferred that the toxicity-attenuated or -deficient forms of component (a1), assayed by a reference test using this component alone, achieve a reduction of the corresponding activities, such as ADP-ribosylating activity and toxicity parameters, by at least about 98%, more preferably by at least about 99%, and particularly by at least about 99.9% relative to the natural counterpart entire enterotoxin.

In order to have the viral vaccine of the invention exert its full synergistic immunoefficacy for the virus immunogen, component (a1) is combined with component (a2) selected from the group consisting of metal salts and mineral salts. To prepare a fixed and common combination, component (a1) or the virus immunogen component (b), or both, are mixed with component (a2), Suitable examples of component (a2) include, without being limited to, salts of aluminum such as aluminium hydroxide, aluminum oxyhydroxide, aluminium phosphate, aluminum orthophosphate, aluminum hydroxyphosphate, aluminum sulphate, “alum” (potassium aluminium sulphate), as well as salts of iron, zinc and/or calcium. Component (a2) can be present in various forms, for example crystalline, amorphous, as gel and especially hydrogel, as sol, as dispersion, any type of adsorbate, or the like. To further enhance a co-acting relationship between both components (a1) and (a2) in association with the desired viral immunogen, it is preferred that both the component (a1) and the viral immunogen are commonly adsorbed on the metal salt or mineral salt component (a2). Such a common adsorbate is particularly useful for the manufacture of a viral vaccine, especially for use in humans and further preferred for the manufacture of an influenza vaccine.

In an established influenza infection model system, unexpected synergistic effects have been demonstrated by using aluminium hydroxide as a representative example for component (a2) in association with component (a1). Given the general concept of the invention to provide synergy between the specifically selected adjuvant types (a1) and (a2) for the viral immunogen, which is based on a synergy by combining a suitably toxicity-attenuated or -deficient “bacterial danger system” based on enterotoxin subunit A and modified or associated forms thereof as disclosed above, together with a “metal salt or mineral salt based delivery system”, aluminium hydroxide is particularly preferred, while other metal salts and mineral salts can be used as well.

The respective amounts of each of components (a1), (a2) and viral immunogen can be suitably chosen depending on the type of treatment and the patient group, and generally refers to respective amounts per vaccine dose effective to elicit an immune response. As used herein, the specification “eliciting immune response” can be measured, for example, by the generally known hemagglutination assay (HAI). For example, a HAI assay can be performed using the serum samples against the given virus to measure the antibody response. Measurement can for example be performed at day 35.

It is especially beneficial that the present invention allows reducing the adjuvanticity-contributing components (a1) and (a2) to relatively low amount levels, thereby further reducing the risk of possible adverse side effects. Accordingly, it is possible to choose an amount of component (a1) in a range of above 0 μg, preferably at a minimum to provide adjuvant effect of its own, to about 500 μg per vaccine dose. Preferred content levels of component (a1) range from about 1 μg to about 250 μg, more preferably from about 5 μg to about 150 μg, particularly from about 10 μg to about 100 μg. For component (a2), a range of above 0 μg, preferably at a minimum to provide adjuvant effect of its own, to about 1 mg per vaccine dose is well-suited, while a preferred lower dose range from about 10 μg to about 500 μg, particularly from about 50 μg to about 150 μg, especially about 100 μg respectively per vaccine dose is preferred. Appropriate upper limitation of each of components (a1) and (a2) can be beneficially chosen in terms of control of, and lowering the risk of adverse side effects.

The viral immunogen is used in an amount per vaccine dose sufficient and effective to elicit an immune response specifically against a given virus prescribed by the selected viral vaccine. While typically applied immunogen or antigen contents per vaccine dose can be used in the viral vaccines according to the present invention, as respectively known by those skilled in the art and the user, the demonstration of synergistically enhanced immune potentiating effects even when using sub-optimal antigen (HA) doses in ferrets which mimic the situation in humans, well supports the concept of reducing the amount of viral immunogen or antigen to non-typical relatively lower levels. In the case of influenza specific immunogen component and similar situations of other virus vaccines, the content of the respective specific immunogen or antigen is defined and determined by an amount of hemagglutinin (HA) antigen—or in other cases of corresponding viral surface antigens—of about 15 μg per viral strain per vaccine dose or lower, more preferably containing at most about 10 μg, particularly at most about 5 μg and very suitably about 2 μg per viral strain per vaccine dose. Using such normally sub-optimal “low dose” treatment concepts, the present invention allows to adapt to emergency situations such as epidemics and in particular pandemics, as the reduction of antigen content will make higher number of doses available for a given and produced amount of viral antigen. Thus, the new adjuvant concept according to the present invention allows to employ and adapt beneficial antigen sparing mechanisms, for example in cases of certain viral outbreaks. This makes the vaccine of the present invention an ideal candidate for seasonal pre-pandemic and pandemic vaccines.

Suitable vaccine types, which can be obtained in the present invention, can be selected from subunit vaccines, split vaccines, inactivated whole virus vaccines, attenuated virus vaccines, recombinant protein vaccines, virosome vaccines, virus-like-particle vaccines, and similar and further developed vaccines. Since the new adjuvant concept of the present invention can significantly enhance immunogenicity, the present invention can be beneficially applied to vaccines containing purified antigen preparations like subunit, split and similar vaccines as well as artificially produced virosomses, thereby reducing risks of infections or complications while eliciting a profound immune response.

The advantages of the present invention makes the viral vaccine particularly suitable to provide influenza vaccine and thereby to choose an influenza specific immunogen to be included into the vaccine. The influenza vaccine otherwise is preferably free of non-viral immunogens and especially free of bacterial antigens other than components (a1) and (a2). The influenza specific immunogen can be accomplished by providing whole influenza virus particles, which are inactivated by chemical and/or physical means, or by isolated and/or purified influenza virus antigens. The influenza antigens can be presented by a suitable preparation, for example in the form of a subunit vaccine, prepared e.g. by using suitable detergents. In case of providing influenza vaccine, the viral immunogen is determined by the HA antigen present, and the above indicated amounts correspondingly apply to the measured HA content as the relevant antigen. A typical influenza vaccine would comprise, in case of a mono-valent viral vaccine, about 15 μg HA per vaccine dose, or in case of a multi-valent vaccine an amount of about 15 μg per strain per vaccine dose, that is for a tri-valent vaccine, about 45-50 μg per vaccine dose. The advantages of the present invention are particularly useful in the lower, normally “sub-optimal” HA antigen range substantially below the above levels as described above, i.e. containing at most about 10 μg, particularly at most about 5 μg and very suitably about 2 μg per viral strain per vaccine dose. This aspect is especially useful to provide a seasonal, pre-pandemic and pandemic influenza vaccine.

The influenza specific immunogen is beneficially chosen from the influenza antigen classes H1, H2, H3, H5, H6, H7, N1, N2, N3 or N7, either alone or in combination, preferably from the influenza antigen classes H1, N1, H2, N2, H3, N2, H6, N1, H7, N3 or H7, N7, preferably by class H3 and H5 and particular by H1N1 or H5N1. The influenza specific immunogen may be characteristic of influenza A or influenza B.

The description above correspondingly applies to the influenza vaccine provided according to the present invention. In a particularly preferred embodiment, the present invention provides an influenza vaccine comprising, as component (a1) a detoxified or non-toxic mutant of subunit A of heat-labile enterotoxin (LTA), and as component (a2) at least one substance selected from the group consisting of metal salts and mineral salts; and an influenza-specific antigen, notably hemagglutinin (HA) and optionally neuraminidase (NA).

Besides influenza, other suitable immunogen components for the viral vaccines according to the present invention are defined by an antigen of the respectively relevant pathogen to be immunized or treated. This includes, without being limited to, immunogens derived from viruses selected from the group consisting of hepatitis B, hepatitis A, hepatitis C, hepatitis D & E virus, Non-A/Non-B Hepatitis virus, pox and smallpox viruses, polio virus, measles virus, human immunodeficiency virus (HIV), enteroviruses, retroviruses, respiratory syncytial virus, rotavirus, human papilloma virus, varicella-zoster virus, yellow fever virus, SARS virus, animal viruses, herpes viruses, cytomegalovirus, varicella zoster, Epstein Barr virus, para-influenza viruses, adenoviruses, coxsakie viruses, picorna viruses, rhinoviruses, rubella virus, papovirus, and mumps virus. Some non-limiting examples of known viral antigens other than the Influenza virus antigens mentioned above may include the following: antigens derived from HIV-I such as tat, nef, gpl20 or gpl[beta]O, gp40, p24, gag, env, vif, vpr, vpu, rev or part and/or combinations thereof; antigens derived from human herpes viruses such as gH, gL gM gB gC gK gE or gD or or part and/or combinations thereof or Immediate Early protein such as ICP27, ICP47, ICP4, ICP36 from HSVI or HSV2; antigens derived from cytomegalovirus, especially human cytomegalovirus such as gB or derivatives thereof; antigens derived from Epstein Barr virus such as gp350 or derivatives thereof; antigens derived from Varicella Zoster Virus such as gp I, 11, 111 and IE63; antigens derived from a hepatitis virus such as hepatitis B, hepatitis C or hepatitis E virus antigen (e.g. env protein EI or E2, core protein, NS2, NS3, NS4a, NS4b, NS5a, NS5b, p7, or part and/or combinations thereof of HCV); antigens derived from human papilloma viruses (for example HPV6, 11, 16, 18, e.g. LI, L2, EI, E2, E3, E4, E5, E6, E7, or part and/or combinations thereof); antigens derived from other viral pathogens, such as Respiratory Syncytial virus (e.g F and G proteins or derivatives thereof), parainfluenza virus, measles virus, mumps virus, flaviviruses (e. g. Yellow Fever Virus, Dengue Virus, Tick-borne encephalitis virus, Japanese Encephalitis Virus) or part and/or combinations thereof.

The viral vaccine and influenza vaccine disclosed above have demonstrated beneficially high immunogenicity when the vaccine is used in the treatment including intramuscular administration. The dosage form of the viral vaccine according to the present invention may additionally contain suitable carriers and excipients generally known for vaccine formulations, such as, alone or together, buffers, salts, stabilizers, preservatives, aqueous or other solvent media, injection fluids, etc.

The present invention is described in further detail by the following non-limiting examples.

### Comparative Experiments Using Alum and Subunit B of Heat Labile Enterotoxin (LTB)

An experimental study was designed to determine effects of respective adjuvant candidates aluminium hydroxide and LTB in a ferret model. The ferret influenza infection model closely mimics infection in humans, it is an established animal model for the study of Influenza infection (van der Laan et al, Expert Rev. Vaccines 7(6), 783-793 (2008); Chen et al., 1995; Boyd & Beeson, 1975; Scheiblauer et al., 1995; Sweet & Smith, 1980; Toms et al., 1977), and has previously been used to determine the efficacy of Influenza vaccines (Fenton et al., 1981; Webster et al., 1994). “It remains the best available model for human Influenza”, see The ferret as an animal model of Influenza virus infection. C. Sweet, R. J. Fenton and G. E. Price Handbook of Animal Models of Infection, 1999.

The ferrets were initially primed with Influenza A/Panama/2007/99 (H3N2) virus via the intranasal route, and then immunised twice with test or control articles. HA was monovalent and derived from influenza A/New Caledonia/20/99.
15 μg HA/dose (30 μg HA/ml)
1.7 μg HA/dose (3.3 μg HA/ml) and 500 μg Alum (1000 μg Alum/ml)
1.7 μg HA/dose (3.3 μg HA/ml) and 5 μg LTB (10 μg LTB/ml.

Each dose consisted of 500 μl, administered as 1×250 μl i.m. per hind limb, and the animals were vaccinated on two occasions.

Three weeks after second vaccination 10 animals per treatment group were challenged with live Influenza A/New Caledonia/20/99 (H1N1) virus.

Post-challenge, all animals were monitored for weight, temperature and health score, so as to determine the immunogenicity and protective efficacy of the test and control articles. Nasal washes were performed on the animals on days 1-6, and the samples for days 1-4 were analysed for viral shedding from the nasal mucosa. Serum samples were collected on the priming, vaccination, challenge, and culling days to assess seroconversion.

When the test and control article treated groups were compared to the positive control article treated group (which was primed and vaccinated with 15 μg HA per dose, but did not contain an adjuvant) by ANOVA and/or non-parametric Wilcoxon's rank-sum test, the differences between group parameters below were observed following challenge with Influenza A/New Caledonia/20/99 virus (H1N1):
only the alum-adjuvanted group (1.7 μg HA and 500 μg Alum) was observed to have a statistically significant reduction in mean maximum viral titre in nasal washes (p=<0.001); and
only the alum-adjuvanted group (1.7 μg HA and 500 μg Alum) was observed to have a statistically significant reduction in mean sum of viral titre in nasal washes (p=<0.001).

On the other hand, the LTB test article treated group did not show a statistically significant reduction in mean maximum or mean sum viral titre.

None of the groups were observed to have a statistically significant reduction in mean maximum symptom score, a statistically significant reduction in mean maximum weight loss, or a statistically significant reduction in mean maximum temperature rise, respectively following challenge with Influenza A/New Caledonia/20/99 virus (H1N1).

### Examples 1 to 4 and Comparative Examples 1 to 6

An experimental study was designed to determine the immunopotentiating effects of detoxified or non-toxic mutant of LTA adjuvant and aluminium hydroxide adjuvant respectively alone or in combination, using the ferret model mentioned in the above Comparative Experiments. A H5N1 subunit influenza vaccine was used for challenging the ferrets.

This study design is a useful model for the development of a pandemic Influenza vaccine and accepted in the framework of a ‘core dossier’ for a pandemic vaccine because an actual pandemic strain is not available

Outbred, fitch or albino, female ferrets were used as a test system. 100 Ferrets (aged about 5-7 months; 700-1200 g bodyweight at day 0, having been feeded with High Density Ferret LabDiet) were subjected to vaccination (10 groups of 10). Animals were identified by transponders (IPTT-300; Bio Medic Data Systems, Inc., USA).

Vaccine administration dosing were given at days 0 and 14. Body weights were taken at days 0, 7, 14, 21, 28, and 35, and serum samples were taken for HAI test at days 0, 14, and 35. The influenza vaccines (Surface Antigen, Inactivated, Prepared in Cell Cultures) were derived from NIBRG-14 (National Institute for Biological Standards and Control, Hertfordshure, GB) as an A/Vietnam/1194/04 (H5N1)-like strain.

Vaccines were formulated with and without the addition of adjuvants. Adjuvants used were aluminum hydroxide alone at a high and low dose, non-toxic mutant LTA alone at a high and low dose and a combination of the two adjuvants at high and low doses of each. Non-toxic mutant LTA is a genetically altered form of the A-subunit of the HLT (Heat Labile Toxin) holoenzyme and as such contains the mutation R192G (in the following briefly denoted as “LTA”).

One vaccine dose of formulated vaccine consisted of 500 μl, which was administered as 1×250 μl per hind limb (intramuscularly). The animals were vaccinated on two separate occasions.

The tested vaccines are specifically identified in the following. It is noted that the total amounts of the respective ingredients per vaccine dose corresponds to half of the indicated concentration values, as a total dose volume of 0.5 ml was respectively administered. Solution medium is aqueous PBS.

### Control Vaccine 1 (Comparative Example 1)

30 μg HA/ml;

### Control Vaccine 2 (Comparative Example 2)

4 μg HA/ml;

### Reference Vaccine 3 (Comparative Example 3)

4 μg HA/ml+0.2 mg/ml aluminium hydroxide;

### Reference Vaccine 4 (Comparative Example 4)

4 μg HA/ml+1.0 mg/ml aluminium hydroxide;

### Reference Vaccine 5 (Comparative Example 5)

4 μg HA/ml+0.02 mg/ml LTA;

### Reference Vaccine 6 (Comparative Example 6)

4 μg HA/ml+0.20 mg/ml LTA;

### Test Vaccine 7 According to the Invention (Example 1)

4 μg HA/ml+0.2 mg/ml aluminium hydroxide+0.02 mg/ml LTA;

### Test Vaccine 8 According to the Invention (Example 2)

4 μg HA/ml+0.2 mg/ml aluminium hydroxide+0.20 mg/ml LTA;

### Test Vaccine 9 According to the Invention (Example 3)

4 μg HA/ml+1.0 mg/ml aluminium hydroxide+0.02 mg/ml LTA;

### Test Vaccine 10 According to the Invention (Example 4)

4 μg HA/ml+1.0 mg/ml aluminium hydroxide+0.20 mg/ml LTA.

All test vaccines contained thiomersal as preservative (100 μg/ml)

For analysis, collected whole blood samples were centrifuged at 3000 rpm for 10 minutes. The serum was then decanted appropriately into fresh tubes and stored at −20° until analysed. The HAI assay was performed upon the serum samples collected at day 0 and 35 only against Influenza NIBRG-14 (H5N1) virus to measure the antibody response.

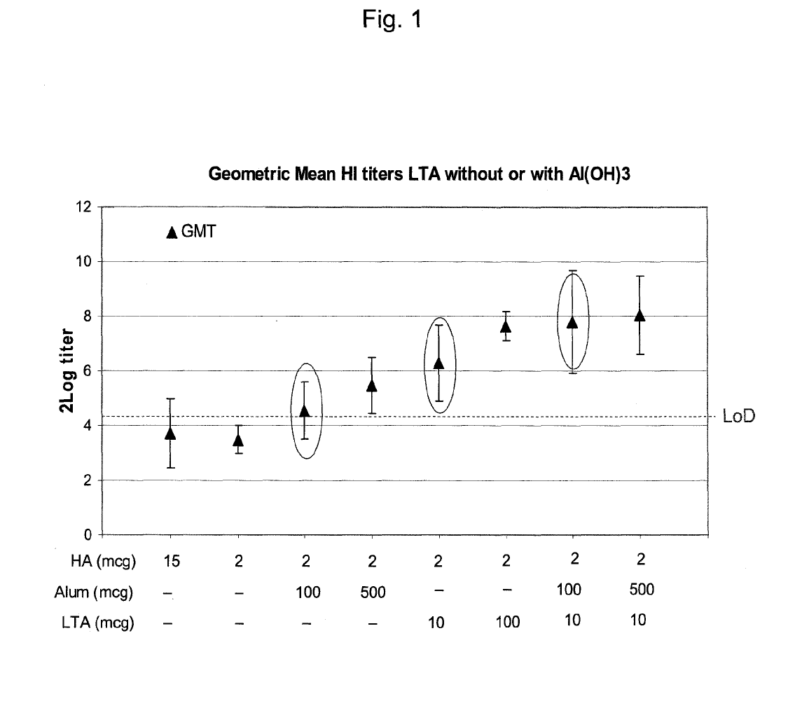

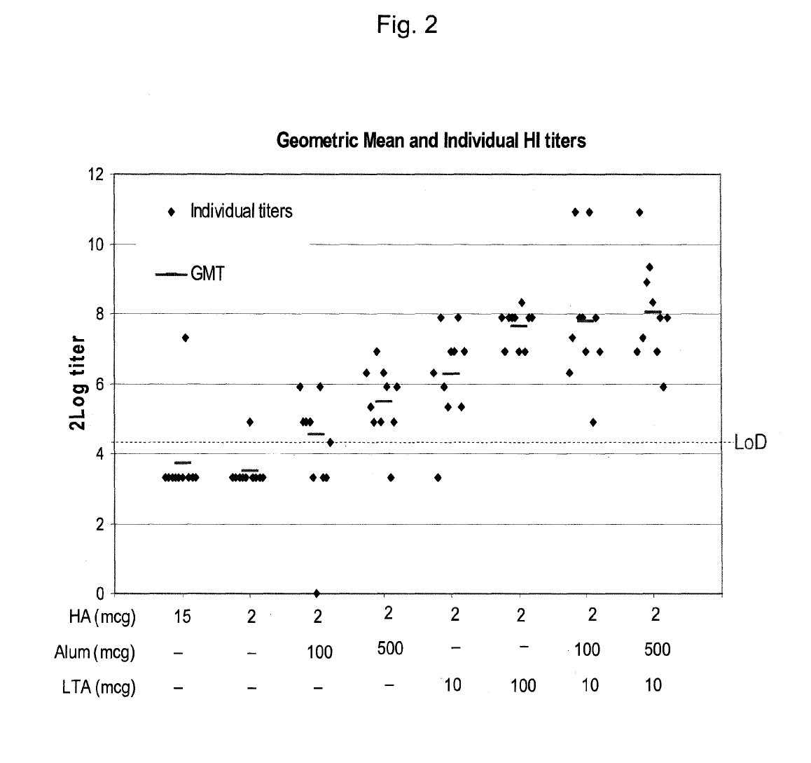

It was observed that vaccines comprising virus antigen even at a low dose per vaccine (inactivated sub unit influenza antigen with 2 μg HA) but adjuvanted with both non-toxic mutant LT-A (10 μg) and Al(OH)₃ (100 μg) induced HI titer that were significantly higher than for non-adjuvanted vaccine with a dose level of 15 μg HA or vaccines at a dose level of 2 μg HA and adjuvanted with 10 μg LT-A only or 100 or 500 μg Al(OH)₃ only (see FIG. 1 and FIG. 2; mcg=μg). Surprisingly, given the same amount of virus immunogen (2 μg HA) the adjuvant combination exerted a specific immune response exceeding the additive magnitude when using each adjuvant alone, as indicated by the resulting logarithmic titer values, thereby demonstrating a true synergistic effect. Moreover, the results demonstrate that, owing to this synergistic effect exerted even at relatively low amounts of virus antigen, non-toxic mutant LTA and Al(OH)₃ respectively—i.e. amounts which otherwise, if considered alone, may fall in “sub-optimal” ranges—control and limitation of the respective active vaccine ingredients becomes possible, if desired. Therefore, while the new adjuvant concept works with viral antigens at low dose levels, on the contrary high doses of each of virus antigen (HA), non-toxic mutant modified LTA adjuvant and aluminum adjuvant respectively would be required to elicit an acceptable immune response.

Thus, the vaccine according to the present invention can, for example, be useful as an antigen sparing concept, as an attractive concept for seasonal, epidemic and pandemic vaccines, as well as for the purpose of reducing risks of adverse side effects. All these features are particularly beneficial for influenza vaccine concepts.

### APPLICABILITY

As becomes apparent, the combination of detoxified or non-toxic mutant of subunit A of an AB type exotoxin with an aluminium salt can be used to provide a substantial antigen-sparing effect of at least one influenza-specific antigen against influenza virus, relative to the use of the corresponding type and amount of influenza-specific antigen which elicits about the same or a lower magnitude of immune response against said at least one influenza-specific antigen of influenza virus but uses neither said subunit A nor said aluminium salt. Since the obtained data demonstrate that even a 7.5-fold reduced antigen (HA) still elicited a substantially higher immune response in case of the combination (doubled log titer), it can be expected that an at least 3-fold, preferably an at least 5-fold and even more preferably a 10-fold antigen-sparing effect can be achieved with the inventive combination.

Moreover, it becomes further apparent that the combination of detoxified or non-toxic mutant of subunit A of an AB type exotoxin with an aluminium salt can be used to provide a remarkably increased immune response against at least one influenza-specific antigen of influenza virus, relative to the use of the corresponding type and amount of influenza-specific antigen which neither uses said subunit A nor said aluminium salt. Since the obtained data demonstrate that with the same low level of antigen content (2 μg HA per dose) an even 2 log titer increase is obtained, it can be expected that an at least 20-fold, preferably an at least 50-fold and even more preferably a 100-fold higher immune response can be achieved with the inventive combination.

1. An influenza vaccine comprising:
a detoxified or non-toxic mutant of subunit A of heat-labile enterotoxin (HLT) of Escherichia coli;
at least one aluminium salt; and
at least one influenza-specific antigen.

2. The influenza vaccine according to claim 1, wherein said detoxified or non-toxic mutant of subunit A has reduced ADP-ribosylating activity.

3. The influenza vaccine according to claim 1, wherein said at least one aluminium salt is selected from the group consisting of aluminum hydroxide, aluminum phosphate and alum.

4. The influenza vaccine according to claim 1, wherein said at least one aluminium salt is present in an amount of up to about 0.5 mg per vaccine dose.

5. The influenza vaccine according to claim 1, wherein said at least one influenza-specific antigen comprises hemagglutinin (HA), which is limited by an amount of up to about 15 μg HA per viral strain per vaccine dose.

6. The influenza vaccine according to claim 5, wherein said vaccine also comprises influenza neuraminidase (NA).

7. The influenza vaccine according to claim 1, which is a seasonal, pre-pandemic, or pandemic influenza vaccine.

8. The influenza vaccine according to claim 1, wherein the influenza-specific antigen is defined by the influenza antigen class H1, H2, H3, H5, H6, H7, N1, N2, N3 or N7, either alone or in combination.

9-13. (canceled)

14. A method of immunization comprising administering an influenza vaccine comprising a detoxified or non-toxic mutant of subunit A of heat-labile enterotoxin (HLT) of Escherichia coli and an aluminium salt:
wherein said vaccine provides an at least 3-fold antigen-sparing effect of at least one influenza-specific antigen against influenza virus, relative to the use of the corresponding type and amount of influenza-specific antigen which elicits about the same or a lower magnitude of immune response against said at least one influenza-specific antigen of influenza virus but uses neither said subunit A nor said aluminium salt.

15. The method of claim 14, wherein said influenza vaccine further comprises at least one influenza-specific antigen.

16. An adsorbate comprising
at least one influenza-specific antigen, and
a detoxified or non-toxic mutant of subunit A of heat-labile enterotoxin (HLT) of Escherichia coli,
respectively adsorbed on aluminum hydroxide.

17. The adsorbate according to claim 16, wherein said at least one influenza-specific antigen comprises influenza hemagglutinin (HA).

18. A method for immunizing a patient against influenza virus infection, comprising:
administering to a person in need thereof a dose of at least one influenza-specific antigen, against which the patient shall be immunized,
administering to said person at least one aluminium salt, and
administering to said person a detoxified or non-toxic mutant of subunit A of heat-labile enterotoxin (HLT) of Escherichia coli,
wherein the respective administration steps are carried out simultaneously, or independently from each other within one or separate dosage forms, or sequentially in a prescribed order.

19. The method according to claim 18, wherein said at least one influenza-specific antigen comprises influenza hemagglutinin (HA), which is limited by an amount of up to about 15 μg HA per viral strain per vaccine dose.

20. The method according to claim 19, wherein said hemagglutinin is in an amount of about 2 μg HA per viral strain per vaccine dose.

21. The method according to claim 18, wherein said at least one influenza-specific antigen comprises influenza hemagglutinin (HA) in combination with influenza neuraminidase (NA).

22. The method according to claim 18, wherein said at least one aluminium salt comprises aluminum hydroxide.

23. The method according to claim 18, wherein at least one of the respective administration steps is by intramuscular injection.

24. The influenza vaccine according to claim 3, wherein said at least one aluminium salt is aluminum hydroxide.

25. The influenza vaccine according to claim 5, wherein said at least one influenza-specific antigen comprises hemagglutinin (HA) in an amount of about 2 μg HA per viral strain per vaccine dose.

26. The influenza vaccine according to claim 8, wherein the influenza-specific antigen is defined by the influenza antigen class H1N1, H2N2, H3N2, H5N1, H6N1, H7N3 or H7N7.

27. The influenza vaccine according to claim 26, wherein the influenza-specific antigen is defined by the influenza antigen class H1N1 or H5N1.

28. The adsorbate according to claim 16, wherein said at least one influenza-specific antigen comprises influenza hemagglutinin (HA) in combination with influenza neuraminidase (NA).
